(19) United States
(12) Patent Application Publication
(10) Pub. No.: US 2026/0232916 A1
(43) Pub. Date: Aug. 13, 2026

(54) A CAP ASSEMBLY OF A MEDICAMENT DELIVERY DEVICE

(71) Applicant: SHL Medical AG, Zug (CH)

(72) Inventors: Alexander Stiegler, Taoyuan (TW); Lucas Carpenter, Taoyuan (TW); Fuhan Hsieh, Taoyuan (TW); Ming-Ting Yin, Taoyuan (TW); Ali Bakhtiyari, Taoyuan City (TW)

(21) Appl. No.: 19/152,265

(22) PCT Filed: Jan. 25, 2024
(86) PCT No.: PCT/EP2024/051795

§ 371 Date: Jul. 30, 2025

(30) Foreign Application Priority Data
Feb. 8, 2023 (EP) 23155570.7

(51) Int. Cl. A61M 5/32; A61M 5/31

(52) U.S. Cl. CPC A61M 5/3202; A61M 2005/3104; A61M 2205/276; A61M 2205/8206

(57) ABSTRACT

The present disclosure provides a cap assembly of a medicament delivery device. The cap assembly includes a main body extending along a longitudinal axis between a proximal end and a distal end. The main body is configured to be removably attached to a housing of the medicament delivery device so that a delivery member of the medicament delivery device is covered by the main body before use. The main body comprises a user-accessible outer surface wherein the main body comprises a distally directed surface. The cap assembly further includes a secondary body placing on a part of the user-accessible outer surface of the main body. The secondary body comprises a proximally directed surface. The secondary body is movable relative to the main body in the direction of the longitudinal axis between a distal position where the proximally directed surface of the secondary body is spaced apart from the distally directed surface of the main body and a proximal position where the proximally directed surface of the secondary body is engaged with the distally directed surface of the main body.

### TECHNICAL FIELD

The present disclosure generally relates to a cap assembly of a medicament delivery device, and particularly to a cap assembly of a medicament delivery device comprising a secondary body.

### BACKGROUND

Medicament delivery devices such as auto-injectors, inhalers, or on-body devices are generally known for the self-administration of a medicament by patients without formal medical training. For example, patients suffering from diabetes or people who are undergoing an artificial fertilization procedure may require repeated injections of insulin or hormone. Other patients may require regular injections of other types of medicaments, such as a growth hormone.

As those medicament delivery devices are designed for patients without formal medical training and operation of those medicament delivery devices might be taking place in a patient's own house, which is usually not in a place of professional health/medical care, e.g. hospital, clinic or health centres, there is a demand for automatically recording every single delivery operation that has been taken by the user. The record can help the user to track his/ her medicament intake or as the basis of an alarm as the next operation reminder; the record can also help a medical doctor or a health care provider to track the compliance of the user regarding the therapeutic regimen. Furthermore, to ensure users' safety in accessing/using a medicament delivery device, there is also a demand for stopping users from accessing or using a medicament delivery device which has been used.

Electronic medicament delivery devices have been developed for allowing patients themselves to safely administer medicament, without the need for help from health professionals, and for allowing transmission of data to the health professionals. Data is generally transmitted by an electronic component that is powered by a battery integrated within the device or through a wired connection by an external power source.

Nowadays, the material cost of electronics that can provide the above-mentioned functions is reducing. However, the cost of manufacture is increasing, by either the increasing cost of manpower or the increasing cost of making a more complicated automatic assembling machine, so there is still room for simplifying the assembling process for medicament delivery devices.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a cap assembly of a medicament delivery device, the cap assembly comprising: a main body extending along a longitudinal axis between a proximal end and a distal end; wherein the main body is configured to be removably attached to a housing of the medicament delivery device such that a delivery member of the medicament delivery device is covered by the main body before use; wherein the main body comprises a user-accessible outer surface extending around the longitudinal axis; wherein the main body comprises a distally directed surface; a secondary body placed on a part of the user-accessible outer surface of the main body; wherein the secondary body comprises a proximally directed surface; and wherein the secondary body is movable relative to the main body in the direction of the longitudinal axis between a distal position where the proximally directed surface of the secondary body is spaced apart from the distally directed surface of the main body and a proximal position where the proximally directed surface of the secondary body is engaged with the distally directed surface of the main body.

Preferably, according to another embodiment, the user-accessible outer surface of the main body is fully enclosed by the secondary body when the secondary body is in the distal position.

Preferably, according to another embodiment, the secondary body comprises a distally directed surface engaged with a proximally directed surface of the main body when the secondary body is in the distal position.

Preferably, according to another embodiment, the secondary body comprises an activator configured to activate a switch of a set of electronics when the secondary body is in the proximal position.

Preferably, according to another embodiment, the switch is configured to be activated by being moved from an inactive state to an active state.

Preferably, according to another embodiment, the set of electronics is attached to the secondary body, the main body, or the housing of the medicament delivery device.

Preferably, according to another embodiment, the set of electronics is attached to the secondary body.

Preferably, according to another embodiment, the activator is a part of the switch.

Preferably, according to another embodiment, the switch is in the inactive state when the secondary body is in the distal position.

Preferably, according to another embodiment, the switch is in the active state when the secondary body is in the proximal position.

Preferably, according to another embodiment, the switch comprises a circuit connected to a battery.

Preferably, according to another embodiment, the activator is a portion of the circuit that is interrupted by an insulating sheet when the switch is in the inactive state and the circuit is connected to the battery when the switch is in the active state.

Preferably, according to another embodiment, the insulating sheet is attached to either the housing of the medicament delivery device or the main body of the cap assembly such that the insulating sheet is in contact with the circuit when the secondary body is in the distal position.

Preferably, according to another embodiment, the insulating sheet is spaced apart from the circuit when the secondary body is in the proximal position.

Alternatively, according to another embodiment, the insulating sheet could be directly put between the battery and the electronics to interrupt the circuit (no switch needed).

Preferably, according to another embodiment, the insulating sheet is placed between the PCB contact and the battery pole.

Alternatively, according to another embodiment, the switch comprises a distally directed surface.

Preferably, according to another embodiment, the distally directed surface of the switch is configured to be engaged with a part of the housing of the medicament delivery device or a part of the main body of the cap assembly when the secondary body is in the distal position such that the switch is in its inactive state when the secondary body is in the distal position. Preferably, according to another embodiment, the distally directed surface of the switch is the distally directed surface of the secondary body.

Preferably, according to another embodiment, the switch comprises two conductive parts and a biasing member.

Preferably, according to another embodiment, the biasing member is configured to bias at least one of the conductive parts of the switch to be in contact with the other one of the conductive parts of the switch.

Preferably, according to another embodiment, when the distally directed surface of the switch is engaged with either the part of the housing of the medicament delivery device or the part of the main body, the distally directed surface of the switch is configured to be pressed one of the conductive parts against the biasing member by the part of the housing of the medicament delivery device or the part of the main body such that the two conductive parts are spaced apart from one another.

Alternatively, according to another embodiment, the switch comprises a reed switch; wherein the main body or the housing of the medicament delivery device comprises a magnet.

Preferably, according to another embodiment, the secondary body is formed by two parts that are attached to one another by being moved in the direction transverse to the longitudinal axis during manufacture process.

Preferably, according to another embodiment, the set of electronics are positioned between two parts of the secondary body.

Preferably, according to another embodiment, the set of electronics is attached to the main body.

Preferably, according to another embodiment, the activator is the distally directed surface of the secondary body; and wherein the switch comprises the proximally directed surface of the main body.

Preferably, according to another embodiment, the switch comprises two conductive parts and a biasing member.

Preferably, according to another embodiment, the biasing member is configured to bias at least one of the conductive parts of the switch to be in contact with the other one of the conductive parts of the switch.

Preferably, according to another embodiment, when the distally directed surface of the secondary body is engaged with the proximally directed surface of the switch, the distally directed surface of the secondary body is configured to press one of the conductive parts against the biasing member such that the two conductive parts are spaced apart from one another.

Alternatively, according to another embodiment, the switch comprises a reed switch; wherein the secondary body comprises a magnet.

Preferably, according to another embodiment, the secondary body comprises a ring-shaped section movably connected to the main body.

Preferably, according to another embodiment, the secondary body is tubular.

Preferably, according to another embodiment, the cap assembly is used in a sub-assembly of a medicament delivery device.

Preferably, according to another embodiment, the sub-assembly comprises the housing of the medicament delivery device.

Preferably, according to another embodiment, the set of electronics is attached to the housing.

Preferably, according to another embodiment, the switch is adjacent to the secondary body when the secondary body is in the distal position.

Preferably, according to another embodiment, the switch is spaced apart from the secondary body when the secondary body is in the proximal position.

Preferably, according to another embodiment, the activator is a distally directed surface of the secondary body.

Preferably, according to another embodiment, the switch comprises a proximally directed surface engaged with the distally directed surface of the secondary body when the secondary body is in the distal position.

Preferably, according to another embodiment, the proximally directed surface of the switch is disengaged from the distally directed surface of the secondary body when the secondary body is in the proximal position.

Preferably, according to another embodiment, the switch comprises two conductive parts and a biasing member.

Preferably, according to another embodiment, the biasing member is configured to bias at least one of the conductive parts of the switch to be in contact with the other one of the conductive parts of the switch.

Preferably, according to another embodiment, when the distally directed surface of the secondary body is engaged with the proximally directed surface of the switch, the distally directed surface of the secondary body is configured to press one of the conductive parts against the biasing member such that the two conductive parts are spaced apart from one another.

Alternatively, according to another embodiment, the switch comprises a circuit connected to a battery.

Preferably, according to another embodiment, the activator is a portion of the circuit that is interrupted by an insulating sheet when the switch is in the inactive state and the circuit is connected to the battery when the switch is in the active state.

Preferably, according to another embodiment, the insulating sheet is attached to the secondary body of the cap assembly such that the insulating sheet is in contact with the circuit when the secondary body is in the distal position.

Preferably, according to another embodiment, wherein the insulating sheet is spaced apart from the circuit when the secondary body is in the proximal position.

Alternatively, according to another embodiment, the switch comprises a reed switch; wherein the secondary body comprises a magnet.

Alternatively, according to another embodiment, the sub-assembly comprises a second set of electronics attached to the secondary body of the cap assembly.

Preferably, according to another embodiment, the set of electronics of the housing is electrically connected to the second set of electronics when the main body of the cap assembly is at least partially attached to the housing.

Preferably, according to another embodiment, the set of electronics comprises a biasing member configured to bias the switch from the inactive state to the active state.

Preferably, according to another embodiment, the proximally directed surface of the housing is configured to press the switch against the biasing force from the biasing member when the proximally directed surface of the housing is engaged with the distally directed surface of the switch.

Preferably, according to another embodiment, the switch is pivotal between the inactive state and the active state.

Preferably, according to another embodiment, the switch is pivotal on a plane parallel to the longitudinal axis.

Preferably, according to another embodiment, the set of electronics comprises a circuit and a processor electrically connected to the switch.

Preferably, according to another embodiment, the set of electronics comprises a communication unit connected to the circuit.

Preferably, according to another embodiment, the switch is configured to close the circuit when the switch is in the active state.

Preferably, according to another embodiment, the communication unit is configured to wirelessly send out and/or receive a signal when the switch is activated.

Preferably, according to another embodiment, the set of electronics comprises a battery electrically connected to the circuit via the switch.

Preferably, according to another embodiment, the communication unit is configured to wirelessly send out and/or receive a signal when the circuit is connected to the battery.

Preferably, according to another embodiment, the cap assembly is used in a sub-assembly of the medicament delivery device; the sub-assembly comprises the housing of the medicament delivery device.

Preferably, according to another embodiment, the sub-assembly comprises a delivery member guard telescopically arranged relative to the proximal end of the housing. The delivery member guard is configured to surround the medicament delivery member once the cap body is removed from the housing.

Preferably, according to another embodiment, the sub-assembly is used in a medicament delivery device. The medicament delivery device comprises a medicament container containing medicament, and a medicament delivery member operably connected to the medicament container for delivering the contained medicament.

Preferably, according to another embodiment, the medicament delivery member of the medicament delivery device is a needle or a spray nozzle.

Preferably, according to another embodiment, the medicament container of the medicament delivery device is a syringe, a cartridge or a collapsible bag.

Preferably, according to another embodiment, the medicament container of the medicament delivery device is made of glass material or plastic material.

Preferably, according to another embodiment, the medicament delivery device is an injection device, an inhalation device, or a medical sprayer.

Preferably, according to another embodiment, the medicament delivery device is an auto-injector.

Preferably, according to another embodiment, the medicament delivery device is a hand-held, pen-type auto-injector.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example, pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VelP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament delivery device with a cap assembly of the invention when a secondary body of the cap is in a distal position;

FIG. 2 schematically shows a perspective view of a medicament delivery device with a cap assembly of the invention when the secondary body of the cap is in a proximal position;

FIG. 3 schematically shows a perspective view of the secondary body of FIG. 1.

FIG. 4 schematically shows a perspective view of a medicament delivery device with a cap assembly of the invention in another example when the secondary body of the cap is in the distal position;

FIG. 5 schematically shows a perspective view of a medicament delivery device with the cap assembly of FIG. 4 when the secondary body of the cap is in the proximal position;

FIG. 6 schematically shows a perspective view of a medicament delivery device with the cap assembly of FIG. 4 when a main body of the cap is detached from a housing of the medicament delivery device;

FIG. 7 schematically shows a perspective view of a medicament delivery device with a cap assembly of the invention in another example when the secondary body of the cap is in the proximal position;

FIG. 8 schematically shows a perspective view of a switch.

FIG. 9 schematically shows a perspective view of a medicament delivery device with a cap assembly of the invention in another example when the secondary body of the cap is in the distal position;

FIG. 10 schematically shows a perspective view of a medicament delivery device with the cap assembly of FIG. 9 when the secondary body of the cap is in the proximal position; and

FIG. 11 schematically shows a perspective view of a medicament delivery device with the cap assembly of FIG. 9 when the main body of the cap is detached from the housing of the medicament delivery device.

### DETAILED DESCRIPTION

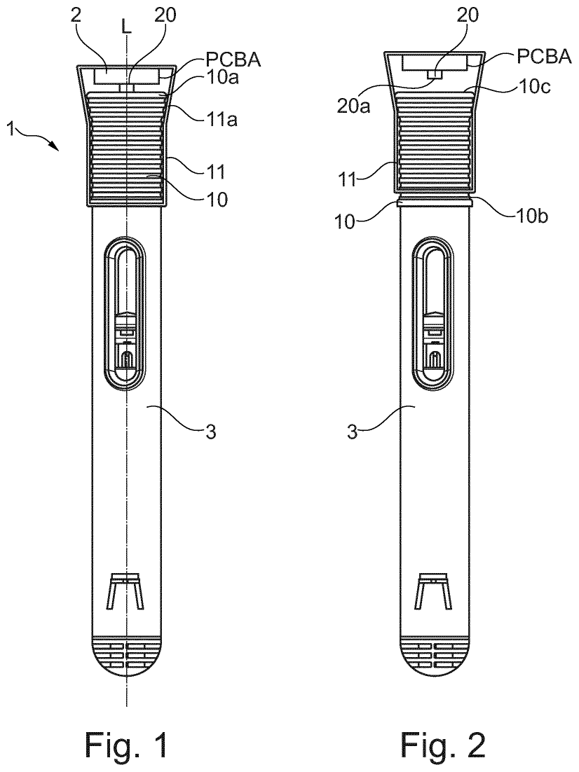

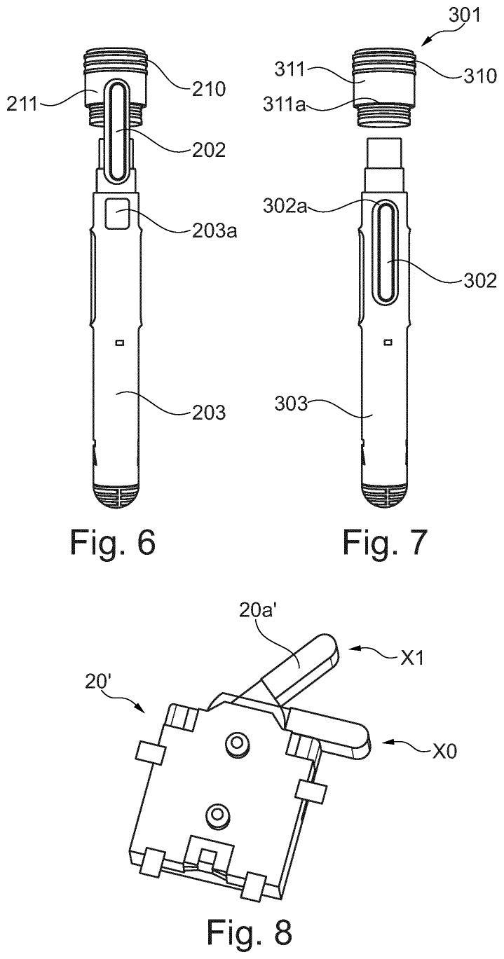

FIGS. 1-11 illustrate cap assemblies 1; 201; 301; 401 of the invention in different examples. The medicament delivery device comprises a medicament container containing a medicament and a delivery member operably connected to the medicament container for delivering the contained medicament. In one example, the medicament container is a syringe, and the delivery member is a needle. In this example, the needle is integral at a proximal end of the syringe. Alternatively, the medicament container can be a cartridge, and the medicament delivery member is a needle that is preassembled to the medicament delivery device and is configured to fluidly communicate with the cartridge during use. Alternatively, the medicament container is a collapsible bag. For example, the medicament delivery device comprises a container carrier. In one example, the medicament container is made of plastic.

This disclosure provides a cap assembly 1; 201; 301; 401 of the medicament delivery device as mentioned above. The cap assembly 1; 201; 301; 401 comprises a main body 10; 210; 310; 410 extending along a longitudinal axis L between a proximal end and a distal end. The main body 10; 210; 310; 410 is configured to be removably attached to a housing 3 of the medicament delivery device so that a delivery member of the medicament delivery device is covered by the main body 10; 210; 310; 410 before use. In one example where the delivery member is sealed by a delivery member cap, a rigid and/or flexible needle shield, or a rubber sheath, for example, the main body comprises a delivery member cap remover. In one example where the medicament container is a syringe, the delivery member is sealed by a combination of a rigid needle shield and a flexible needle shield, the main body comprises a needle shield remover extending from the main body towards the distal end of the main body. The needle shield remover is configured to engage the needle shield such that the needle shield can be removed from the delivery member (a needle of the syringe, in this example), when the cap assembly 1; 201; 301; 401 is removed from the housing 3 of the medicament delivery device. In one example, the needle shield remover is made of metal and is attached to the main body during the production line. Alternatively, the needle shield remover is an integral part of the main body. For example, the main body can be made of plastic material. In this example, the needle shield remover is made of plastic material. In one example, the needle shield remover comprises a gripping arm configured to be engaged with the needle shield. Alternatively, instead of a needle shield remover, the main body seals the delivery member directly.

In one example, the main body 10; 210; 310; 410 is generally cylindrical, as shown in FIGS. 1-11. As many handhold medicament delivery devices, e.g., injection devices, comprise cylindrical housings, the main body of the cap assembly 1; 201; 301; 401 can thus make the medicament delivery device more compact. However, the main body can be any suitable shape depending on the design. For example, if the target patient group commonly has some difficulty with hand gripping, the main body can be shaped with a ring open in a direction transverse to the longitudinal axis or a hook such that the users can easily grip the main body. Alternatively, the main body can have a rectangular or triangle cross-section observed along the longitudinal axis L such that the medicament delivery device can be prevented from rolling on a flat surface when the main body of the cap assembly 1; 201; 301; 401 is attached to the housing 3 of the medicament delivery device.

The main body 10; 210; 310; 410 comprises a user-accessible outer surface 10b extending around the longitudinal axis L, as shown in FIG. 2. In one preferred example, the user-accessible outer surface 10b comprises multiple contiguously connected concave structures and convex structures extending in the direction of the longitudinal axis L. The main body 10; 210; 310; 410 comprises a distally directed surface 10a.

The cap assembly 1; 201; 301; 401 comprises a secondary body 11; 211; 311; 411 placed on a part of the user-accessible outer surface 10b of the main body 10; 210; 310; 410, as shown in FIGS. 1-11. The secondary body 11; 211; 311; 411 comprises a proximally directed surface 11a. The secondary body 11; 211; 311; 411 is configured to be moved by users. The secondary body 11; 211; 311; 411 is movable relative to the main body 10; 210; 310; 410 in the direction of the longitudinal axis L between a distal position where the proximally directed surface 11a of the secondary body 11; 211; 311; 411 is spaced apart from the distally directed surface 10a of the main body 10; 210; 310; 410 and a proximal position where the proximally directed surface 11a of the secondary body 11; 211; 311; 411 is engaged with the distally directed surface 11a of the main body 10; 210; 310; 410. Therefore, the user can remove the cap assembly 1; 201; 301; 401 from the housing 3 by moving the secondary body 11; 211; 311; 411 relative to the main body 10; 210; 310; 410 from the distal position to the proximal position followed by further moving the secondary body 11; 211; 311; 411 together with the main body 10; 210; 310; 410 in the proximal direction until the main body 10; 210; 310; 410 is detached from the housing 3 of the medicament delivery device.

The secondary body 11; 211; 311; 411 can be used as a safety feature that prevents the main body 10; 210; 310; 410 from being accidentally removed from the housing 3 of the medicament delivery device, e.g., by friction. Furthermore, the secondary body 11; 211; 311; 411 can be used as a tamper-evidence. In one example, the secondary body 11 is configured to fully cover the main body 10 of the cap assembly 1, as shown in FIG. 1. In this example, the secondary body 11 is configured to fully enclose the user-accessible outer surface 10b of the main body 10 when the secondary body 11 is in the distal position. In this example, the main body 10 is fully enclosed by the secondary body 11 when the secondary body is in the distal position. In this example, only when the secondary body 11 is moved to the proximal position, a further movement of the secondary body 11 can be transferred to the main body 10, as shown in FIG. 2. As a result, the main body 10 can be removed from the housing 3 of the medicament delivery device. In a preferred example, the secondary body is tubular. Alternatively, the secondary body 211; 311; 411 is configured to partially enclose the main body 210; 310; 410. In one example, the secondary body 211; 311; 411 comprises a ring-shaped section movably connected to the main body 210; 310; 410, as shown in FIGS. 4-11. In this example, as the secondary body 211; 311; 411 extends beyond the main body 210; 310; 410 in the direction transverse to the longitudinal axis L; therefore, when any unintentional force, e.g., friction, applies to the cap assembly 201; 301; 401, it will apply to the secondary body 211; 311; 411 and results in the secondary body 211; 311; 41 moves relative to the main body 210; 310; 410 in the direction of the longitudinal axis L rather than the main body 210; 310; 410 is accidentally detached or loose. In this example, the user can access to a part of the main body 210; 310; 410 before moving the secondary body 211; 311; 411. For example, the user can access a proximal part of the main body 210; 310; 410, as shown in FIGS. 4-11. As a result, the user can either move the secondary body 211; 311; 411 to the proximal position, then further move the secondary body 211; 311; 411 together with the main body 210; 310; 410 away from the housing 3 of the medicament delivery device; or the user can remove the cap assembly from the housing 3 of the medicament delivery device by pulling the main body 210; 310; 410 directly without moving the secondary body 211; 311; 411 to the proximal position first. Alternatively, the cap assembly 201; 301; 401 can be designed to make the user has to move the secondary body 211; 311; 411 first before moving the main body 210; 310; 410. For example, the secondary body comprises a fixture engaged with a counter fixture of the housing when the secondary body is in the distal position and disengaged from the counter fixture of the housing when the secondary body is in the proximal position. It should be noted that instead of a ring-shaped section, the secondary body can comprise an arc-shaped section or a circlip section.

Furthermore, in another example, the secondary body comprises a distally directed surface engaged with a proximally directed surface of the main body when the secondary body is in the distal position. In one example as shown in FIGS. 1-2, the proximally directed surface of the main body 10 is located at the proximal end of the main body 10.

Furthermore, the secondary body can be flexible or rigid. In one example, the secondary body is an elastic ring. In another example, the secondary body is a rigid ring.

In another example, the secondary body 11; 211; 311; 411 comprises an activator 20a; 311a; 411a configured to activate a switch 20; 20′ of a set of electronics 2; 202; 302; 402 when the secondary body 11; 211; 311; 411 is in the proximal position. The switch 20; 20′ is configured to be activated by being moved from an inactive state to an active state. In one example, the set of electronics 2; 202 is attached to the secondary body 11; 211, as shown in FIGS. 1-6. In this example, the activator 20a is a part of the switch 20; 20′. In this example, the switch 20; 20′ is in the inactive state when the secondary body 11; 211 is in the distal position, and the switch 20; 20′ is in the active state when the secondary body 11; 211 is in the proximal position. Alternatively, the set of electronics is attached to the main body. In this example, the activator can be the distally directed surface of the secondary body. In this example, the switch comprises the proximally directed surface of the main body. Alternatively, the set of electronics 302; 402 is attached to the housing 303; 403 of the medicament delivery device as shown in FIGS. 7, 9-11. In this example, the switch is adjacent to the secondary body 311; 411 when the secondary body 311; 411 is in the distal position and the switch is spaced apart from the secondary body 311; 411 when the secondary body 311; 411 is in the proximal position.

In one example, the switch comprises a circuit connected to a battery. The activator is a portion of the circuit that is interrupted by an insulating sheet when the switch is in the inactive state and the circuit is connected to the battery when the switch is in the active state.

In one example where the set of electronics 2; 202 is attached to the secondary body 11; 211, the insulating sheet is attached to either the housing of the medicament delivery device or the main body of the cap assembly such that the insulating sheet is in contact with the circuit when the secondary body 11; 211 is in the distal position; and the insulating sheet is spaced apart from the circuit when the secondary body 11; 211 is in the proximal position.

Alternatively, in one example where the set of electronics 302; 402 is attached to the housing of the medicament delivery device or the main body of the cap assembly, the insulating sheet is attached to the secondary body 311; 411 of the cap assembly such that the insulating sheet is in contact with the circuit when the secondary body is in the distal position and wherein the insulating sheet is spaced apart from the circuit when the secondary body is in the proximal position.

Furthermore, instead of having the insulating sheet, in one example where the set of electronics 2; 202 is attached to the secondary body 11; 211, the switch 20; 20′ comprises a distally directed surface 20a. The distally directed surface of the switch 20; 20′ is configured to be engaged with a part 203a of the housing 203 of the medicament delivery device, as shown in FIGS. 4-6 when the secondary body 202 is in the distal position such that the switch is in its inactive state when the secondary body 202 is in the distal position, as shown in FIG. 4. Alternatively, the distally directed surface 20a of the switch 20 is configured to be engaged with a part of the main body 10 of the cap assembly 1, as shown in FIGS. 1-2 when the secondary body 11 is in the distal position such that the switch 20 is in its inactive state when the secondary body 11 is in the distal position, as shown in FIG. 1. In one example where the secondary body 11 comprises the distally directed surface engaged with the proximally directed surface of the main body 10, preferably, the distally directed surface 20a of the switch 20 is the distally directed surface of the secondary body 11.

Alternatively, in one example where the set of electronics 302; 402 is attached to the housing of the medicament delivery device or the main body of the cap assembly 301; 401, the activator 311a; 411a is a distally directed surface 311a; 411a of the secondary body 311;

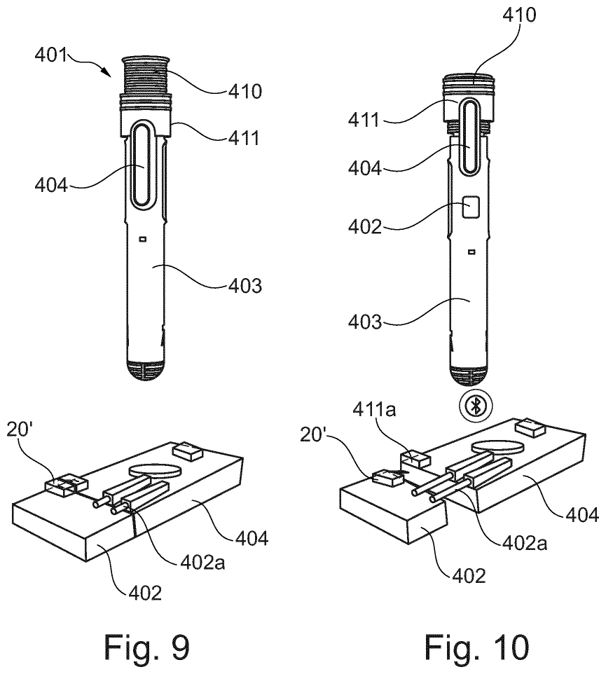

411. In this example, the switch comprises a proximally directed surface engaged with the distally directed surface 311a; 411a of the secondary body when the secondary body 311; 411 is in the distal position, as shown in FIG. 9; and the proximally directed surface of the switch is disengaged from the distally directed surface 311a; 411a of the secondary body 311; 411 when the secondary body is in the proximal position, as shown in FIG. 7 and FIG. 10.

Furthermore, in one example where a surface of the switch is configured to be engaged with another surface in the inactive state, as the examples mentioned above, preferably, the switch comprises two conductive parts and a biasing member. The biasing member is configured to bias at least one of the conductive parts of the switch to be in contact with the other one of the conductive parts of the switch.

In one example where the switch 20; 20′ comprises the distally directed surface 20a; 20′ as mentioned above, when the distally directed surface of the switch 20; 20′ is engaged with either the part of the housing of the medicament delivery device or the part of the main body, the distally directed surface of the switch is configured to be pressed one of the conductive parts against the biasing member by the part of the housing of the medicament delivery device or the part of the main body such that the two conductive parts are spaced apart from one another. In one example, the distally directed surface 20 of the switch 20 is configured to be moved along the longitudinal axis L by the biasing member, as shown in FIGS. 1-2, when the switch is moved from the inactive state to the active state. Alternatively, the distally directed surface 20 of the switch 20 is configured to be moved by the biasing member along a path pivot to a plane that is parallel to the longitudinal axis L, as shown in FIG. 8.

Alternatively, one example where the switch comprises the proximally directed surface as mentioned above, the distally directed surface 311a; 411a of the secondary body 311; 411 is configured to press one of the conductive parts against the biasing member such that the two conductive parts are spaced apart from one another. Similarly, in one example, the proximally directed surface of the switch is configured to be moved along the longitudinal axis L by the biasing member when the switch is moved from the inactive state to the active state. Alternatively, the proximally directed surface of the switch is configured to be moved by the biasing member along a path pivot to a plane that is parallel to the longitudinal axis L.

Alternatively, instead of having a switch that is configured to be moved from an inactive state to an active state by having a surface to be engaged to/disengaged from another surface or removal of an insulating sheet, the switch can be a contactless switch. For example, the switch comprises a reed switch. In one example where the set of electronics is attached to the secondary body 11; 211, the main body or the housing of the medicament delivery device comprises a magnet. Alternatively, in one example where the set of electronics is attached to the main body or the housing of the medicament delivery device, the secondary body comprises a magnet.

In one example, the disclosure provides a sub-assembly comprising the cap assembly 1; 201; 301; 401 as mentioned above, wherein the sub-assembly comprises the housing of the medicament delivery device.

As mentioned above, the secondary body of the cap assembly 1; 201; 301; 401 is configured to be moved to a predetermined position when the user plans to use the medicament delivery device. Thus, the set of electronics can be used to monitor the use of the medicament delivery device. For example, the set of electronics can be used to detect the cap removal of the medicament delivery device. Alternatively, the set of electronics can be used for detecting an unwanted cap removal or the breakage of the sterilization of the delivery member. For example, the set of electronics can be arranged with a proximity sensor or a motion sensor, e.g., an accelerometer or a gyro sensor, such that when the secondary body is in the proximal position, the set of electronics is switched on and start to detect whether the cap assembly is spaced apart from the housing the of medicament delivery device or is moved to a position that the delivery member is no longer in the sterilized sealed state, e.g., the delivery member is not sealed properly. Thus, if the device has been uncapped before the user plans to use the medicament delivery device, the user can be given a warming signal either by the set of electronics directly or by another device that sends a signal based on the detection from the set of electronics.

In a preferred example, the switch 20; 20′ is operable between an active state where a circuit of the electronics is connected to a battery and an inactive state where the circuit is disconnected from the battery. In this example, battery consumption can be minimized.

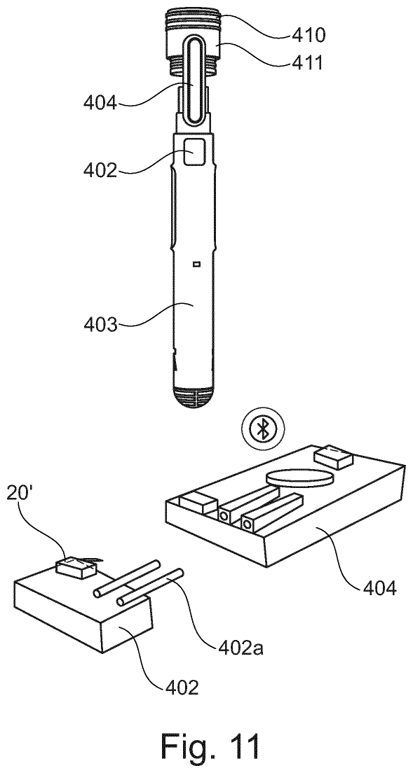

Alternatively, the unwanted cap removal or the breakage of the sterilization of the delivery member can be detected by the sub-assembly comprising a second set of electronics 404 attached to the secondary body 411 of the cap assembly 401. In this example, the set of electronics 402 of the housing 403 is electrically connected to the second set of electronics 404 when the main body 410 of the cap assembly 401 is at least partially attached to the housing 403, as shown in FIGS. 9-10. In this example, the second set of electronics 404 is switched on by an activation signal from the switch of the set of electronics 402 via the electrical connection between the second set of electronics 404 and the set of electronics 402 when the secondary body 411 is in the proximal position. When the main body 410 is detached from the housing 403 of the medicament delivery device, the electrical connection between the second set of electronics 404 and the set of electronics 402 is disconnected, as shown in FIG. 11; therefore, the second set of electronics 404 can detect the cap removal by detecting the disconnection between the second set of electronics 404 and the set of electronics 402.

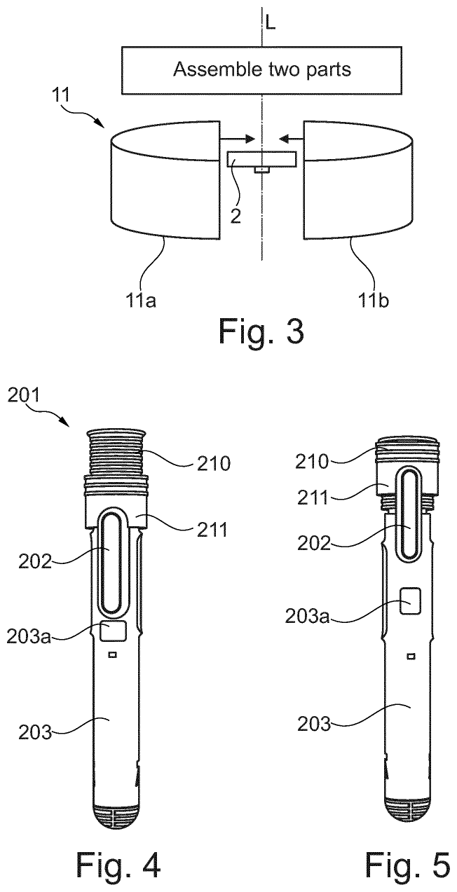

The secondary body of the cap assembly 1 can be attached to the main body of the cap assembly 1 in the production line or can be attached to the main body before the medicament delivery device is given to the end user. For example, a pharmacist can attach the secondary body of the cap assembly 1 to the main body of the cap assembly 1. In a preferred example, the secondary body 11 is formed by two parts 11a, 11b that are attached to one another by being moved in the direction transverse to the longitudinal axis L during the manufacture process, as shown in FIG. 3. As the secondary body is an add-on device to a completed medicament delivery device, the secondary body will not have an impact to the medicament delivery performance of the medicament delivery device or to the sealing of the medicament delivery device. Thus, the manufacturing process of producing medicament delivery devices will not be changed.

In a preferred example, the set of electronics 2 is positioned between two parts of the secondary body 11. In this example, the two parts 11a, 11b of the secondary body 11 can be attached to one another via a snap-fit connection such that the secondary body 11 is attached to the main body 10 by placing the two parts 11a, 11b of the secondary body 11 around the main body 10 of the cap assembly 1 and attaching the two parts 11a, 11b of the secondary body 11 to one another. Alternatively, the secondary body can be coupled to the main body via a magnet buckle (like the anti-thief buckle commonly used for clothes, for example). In this example, the two parts of the secondary body can be attached to one another via the magnet buckle; or the secondary body can be attached to the main body via the magnet buckle. In this example, the secondary body can only be easily detached from the main body when the cap assembly is placed on a specific unlock device. In this example, when the cap assembly comprises the set of electronics (which can be attached to either the secondary body or the main body), the set of electronics can be easily recycled. For example, the end user can dispose of the used cap assembly in a recycling bin and the rest of the medicament delivery device in a medical waste bin. The set of electronics can be detached in the recycling process.

In one example, the set of electronics comprises a communication unit configured to wirelessly send out, e.g., broadcast, and/or receive a signal when the switch is in the active state.

The communication unit can be a short-range communication unit, such as RFID, NFC, infra-red, ZigBee, Bluetooth, Bluetooth beacon, and/or a long-range communication unit, such as 3G, 4G, CAT-M 1, NB-IoT, LoRa, Sigfox, 5G, or GPRS.

In one example, the set of electronics comprises at least one of a processor, e.g., a micro control unit (MCU), a memory, a clock, a communication unit, an indicator and/or a sensor,

In one example, the memory can be a random access memory (RAM), a read-only memory (ROM), an erasable programmable read-only memory (EPROM), an electrically erasable programmable read-only memory (EEPROM) or a Flash memory, such as a compact Flash memory.

In one example, the indicator can be an acoustic indicator, such as a speaker or a buzzer. For example, the indicator can be a visual indicator, such as an e-ink display, an LCD display or a LED light emitter and/or the indicator can be a haptic indicator, such as a vibrator.

In one example, the sensor can be an orientation sensor, such as an accelerometer or a gyroscope, and/or the sensor can be an environmental condition sensor, such as a temperature sensor, vibration sensor or contact sensor.

In one example, the battery is a coin-sized battery or a thin film battery.

Furthermore, the housing, as mentioned in any example, may be provided with (i.e., molded in, molded with) a compound featuring persistently antimicrobial, antifungal, and/or antiviral properties. Alternatively, a compound featuring persistently antimicrobial, antifungal, and/or antiviral properties may be applied to the molded (i.e., finished) components through secondary processes (e.g., chemical vapor deposition), spraying, or dipping processes.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are defined with the clauses below.

1. A cap assembly (1; 201; 301; 401) of a medicament delivery device, the cap assembly (1; 201; 301; 401) comprising:
a main body (10; 210; 310; 410) extending along a longitudinal axis (L) between a proximal end and a distal end; wherein the main body (10; 210; 310; 410) is configured to be removably attached to a housing (3) of the medicament delivery device such that a delivery member of the medicament delivery device is covered by the main body (10; 210; 310; 410) before use; wherein the main body (10; 210; 310; 410) comprises a user-accessible outer surface (10b) extending around the longitudinal axis (L); wherein the main body (10; 210; 310; 410) comprises a distally directed surface (10a);
a secondary body (11; 211; 311; 411) placed on a part of the user-accessible outer surface (10b) of the main body (10; 210; 310; 410); wherein the secondary body (11; 211; 311; 411) comprises a proximally directed surface (11a); and wherein the secondary body (11; 211; 311; 411) is movable relative to the main body (10; 210; 310; 410) in the direction of the longitudinal axis (L) between a distal position where the proximally directed surface (11a) of the secondary body (11; 211; 311; 411) is spaced apart from the distally directed surface (10a) of the main body (10; 210; 310; 410) and a proximal position where the proximally directed surface (11a) of the secondary body (11; 211; 311; 411) is engaged with the distally directed surface (11a) of the main body (10; 210; 310; 410).

2. The cap assembly according to clause 1, wherein the user-accessible outer surface of the main body is fully enclosed by the secondary body when the secondary body is in the distal position.

3. The cap assembly according to clause 1 or 2, wherein the secondary body comprises a distally directed surface engaged with a proximally directed surface of the main body when the secondary body is in the distal position.

4. The cap assembly according to any one of the preceding clauses, wherein the secondary body comprises an activator configured to activate a switch of a set of electronics when the secondary body is in the proximal position; wherein the switch is configured to be activated by being moved from an inactive state to an active state; and wherein the set of electronics is attached to the secondary body, the main body, or the housing of the medicament delivery device.

5. The cap assembly according to clause 4, wherein the set of electronics is attached to the secondary body; wherein the activator is a part of the switch; wherein the switch is in the inactive state when the secondary body is in the distal position; and wherein the switch is in the active state when the secondary body is in the proximal position.

6. The cap assembly according to clause 5, wherein the switch comprises a circuit connected to a battery; wherein the activator is a portion of the circuit that is interrupted by an insulating sheet when the switch is in the inactive state and the circuit is connected to the battery when the switch is in the active state; wherein the insulating sheet is attached to either the housing of the medicament delivery device or the main body of the cap assembly such that the insulating sheet is in contact with the circuit when the secondary body is in the distal position and wherein the insulating sheet is spaced apart from the circuit when the secondary body is in the proximal position.

7. The cap assembly according to clause 5, wherein the switch comprises a distally directed surface; wherein the distally directed surface of the switch is configured to be engaged with a part of the housing of the medicament delivery device or a part of the main body of the cap assembly when the secondary body is in the distal position such that the switch is in its inactive state when the secondary body is in the distal position.

8. The cap assembly according to clause 5 or 7, when dependent on clause 3, wherein the distally directed surface of the switch is the distally directed surface of the secondary body.

9. The cap assembly according to clause 7 or 8, wherein the switch comprises two conductive parts and a biasing member; wherein the biasing member is configured to bias at least one of the conductive parts of the switch to be in contact with the other one of the conductive parts of the switch; wherein when the distally directed surface of the switch is engaged with either the part of the housing of the medicament delivery device or the part of the main body, the distally directed surface of the switch is configured to be pressed one of the conductive parts against the biasing member by the part of the housing of the medicament delivery device or the part of the main body such that the two conductive parts are spaced apart from one another.

10. The cap assembly according to clause 7 or 8, wherein the switch comprises a reed switch; wherein the main body or the housing of the medicament delivery device comprises a magnet.

11. The cap assembly according to any one of the preceding clauses, wherein the secondary body is formed by two parts that are attached to one another by being moved in the direction transverse to the longitudinal axis during manufacture process.

12. The cap assembly according to clause 11 when dependent on clause 5, wherein the set of electronics are positioned between two parts of the secondary body.

13. The cap assembly according to clause 4 when dependent on clause 3, wherein the set of electronics is attached to the main body; wherein the activator is the distally directed surface of the secondary body; and wherein the switch comprises the proximally directed surface of the main body.

14. The cap assembly according to clause 13, wherein the switch comprises two conductive parts and a biasing member; wherein the biasing member is configured to bias at least one of the conductive parts of the switch to be in contact with the other one of the conductive parts of the switch; wherein when the distally directed surface of the secondary body is engaged with the proximally directed surface of the switch, the distally directed surface of the secondary body is configured to press one of the conductive parts against the biasing member such that the two conductive parts are spaced apart from one another.

15. The cap assembly according to clause 13, wherein the switch comprises a reed switch; wherein the secondary body comprises a magnet.

16. The cap assembly according to any one of the preceding clauses, wherein the secondary body comprises a ring-shaped section movably connected to the main body.

17. The cap assembly according to any one of the preceding clauses, wherein the secondary body is tubular.

18. A sub-assembly of a medicament delivery device, the sub-assembly comprising the cap assembly according to any one of the preceding clauses, wherein the sub-assembly comprises the housing of the medicament delivery device.

19. The sub-assembly according to clause 18 when dependent on clause 4, wherein the set of electronics is attached to the housing; wherein the switch is adjacent to the secondary body when the secondary body is in the distal position; and wherein the switch is spaced apart from the secondary body when the secondary body is in the proximal position.

20. The sub-assembly according to clause 19, wherein the activator is a distally directed surface of the secondary body; wherein the switch comprises a proximally directed surface engaged with the distally directed surface of the secondary body when the secondary body is in the distal position; and wherein the proximally directed surface of the switch is disengaged from the distally directed surface of the secondary body when the secondary body is in the proximal position.

21. The sub-assembly according to clause 20, wherein the switch comprises two conductive parts and a biasing member; wherein the biasing member is configured to bias at least one of the conductive parts of the switch to be in contact with the other one of the conductive parts of the switch; wherein when the distally directed surface of the secondary body is engaged with the proximally directed surface of the switch, the distally directed surface of the secondary body is configured to press one of the conductive parts against the biasing member such that the two conductive parts are spaced apart from one another.

22. The sub-assembly according to clause 19 or 20, wherein the switch comprises a circuit connected to a battery; wherein the activator is a portion of the circuit that is interrupted by an insulating sheet when the switch is in the inactive state and the circuit is connected to the battery when the switch is in the active state; wherein the insulating sheet is attached to the secondary body of the cap assembly such that the insulating sheet is in contact with the circuit when the secondary body is in the distal position and wherein the insulating sheet is spaced apart from the circuit when the secondary body is in the proximal position.

23. The sub-assembly according to clause 19 or 20, wherein the switch comprises a reed switch; wherein the secondary body comprises a magnet.

24. The sub-assembly according to any one of clauses 19-23, wherein the sub-assembly comprises a second set of electronics attached to the secondary body of the cap assembly; wherein the set of electronics of the housing is electrically connected to the second set of electronics when the main body of the cap assembly is at least partially attached to the housing.

1-15. (canceled)

16. A cap assembly of a medicament delivery device, the cap assembly comprising:
a main body extending along a longitudinal axis between a proximal end and a distal end, wherein the main body is configured to be removably attached to a housing of the medicament delivery device such that a delivery member of the medicament delivery device is covered by the main body before use, wherein the main body comprises a user-accessible outer surface extending around the longitudinal axis, and wherein the main body comprises a distally directed surface; and
a secondary body placed on a part of the user-accessible outer surface of the main body, wherein the secondary body comprises a proximally directed surface, and wherein the secondary body is movable relative to the main body in the direction of the longitudinal axis between a distal position where the proximally directed surface of the secondary body is spaced apart from the distally directed surface of the main body and a proximal position where the proximally directed surface of the secondary body is engaged with the distally directed surface of the main body.

17. The cap assembly according to claim 16, wherein the user-accessible outer surface of the main body is fully enclosed by the secondary body when the secondary body is in the distal position.

18. The cap assembly according to claim 16, wherein the secondary body comprises a distally directed surface engaged with a proximally directed surface of the main body when the secondary body is in the distal position.

19. The cap assembly according to claim 16, wherein the secondary body comprises an activator configured to activate a switch of a set of electronics when the secondary body is in the proximal position, wherein the switch is configured to be activated by being moved from an inactive state to an active state, and wherein the set of electronics is attached to the secondary body, the main body, or the housing of the medicament delivery device.

20. The cap assembly according to claim 19, wherein the set of electronics is attached to the secondary body, wherein the activator is a part of the switch, and wherein the switch is in the inactive state when the secondary body is in the distal position, and wherein the switch is in the active state when the secondary body is in the proximal position.

21. The cap assembly according to claim 20, wherein the switch comprises a circuit connected to a battery, wherein the activator is a portion of the circuit that is interrupted by an insulating sheet when the switch is in the inactive state and the circuit is connected to the battery when the switch is in the active state, and wherein the insulating sheet is attached to either the housing of the medicament delivery device or the main body of the cap assembly such that the insulating sheet is in contact with the circuit when the secondary body is in the distal position and wherein the insulating sheet is spaced apart from the circuit when the secondary body is in the proximal position.

22. The cap assembly according to claim 20, wherein the switch comprises a distally directed surface, and wherein the distally directed surface of the switch is configured to be engaged with a part of the housing of the medicament delivery device or a part of the main body of the cap assembly when the secondary body is in the distal position such that the switch is in its inactive state when the secondary body is in the distal position.

23. The cap assembly according to claim 20, wherein the distally directed surface of the switch is the distally directed surface of the secondary body.

24. The cap assembly according to claim 16, wherein the secondary body is formed by two parts that are attached to one another by being moved in the direction transverse to the longitudinal axis during manufacture process.

25. The cap assembly according to claim 24, wherein the set of electronics are positioned between two parts of the secondary body.

26. The cap assembly according to 19, wherein the set of electronics is attached to the main body, wherein the activator is the distally directed surface of the secondary body, and wherein the switch comprises the proximally directed surface of the main body.

27. A sub-assembly of a medicament delivery device, the sub-assembly comprising the cap assembly according to claim 16, wherein the sub-assembly comprises the housing of the medicament delivery device.

28. The sub-assembly according to claim 27, wherein the set of electronics is attached to the housing, wherein the switch is adjacent to the secondary body when the secondary body is in the distal position, and wherein the switch is spaced apart from the secondary body when the secondary body is in the proximal position.

29. The sub-assembly according to claim 28, wherein the activator is a distally directed surface of the secondary body, wherein the switch comprises a proximally directed surface engaged with the distally directed surface of the secondary body when the secondary body is in the distal position, and wherein the proximally directed surface of the switch is disengaged from the distally directed surface of the secondary body when the secondary body is in the proximal position.

30. The sub-assembly according to claim 27, wherein the sub-assembly comprises a second set of electronics attached to the secondary body of the cap assembly, and wherein the set of electronics of the housing is electrically connected to the second set of electronics when the main body of the cap assembly is at least partially attached to the housing.
